(19) United States
(12) Patent Application Publication
(10) Pub. No.: US 2026/0215801 A1
(43) Pub. Date: Jul. 30, 2026

(54) TREATMENT SYSTEM

(71) Applicant: FUJIFILM Corporation, Tokyo (JP)

(72) Inventors: Satoru OKADA, Kanagawa (JP); Takeharu NAKAZATO, Kanagawa (JP); Shozo IYAMA, Kanagawa (JP)

(73) Assignee: FUJIFILM Corporation, Tokyo (JP)

(21) Appl. No.: 19/574,454

(22) Filed: Mar. 23, 2026

Related U.S. Application Data
(63) Continuation of application No. PCT/JP2024/019266, filed on May 24, 2024.

(30) Foreign Application Priority Data
Sep. 29, 2023 (JP) 2023-169863

(51) Int. Cl. A61B 17/32; A61B 1/00; A61B 1/018; A61B 17/00

(52) U.S. Cl. CPC A61B 17/320068; A61B 1/00133; A61B 1/018; A61B 2017/0034

(57) ABSTRACT

A treatment system includes a tubular member having a hollow portion therein and having, at one end part, a treatment part provided at an outer periphery of the hollow portion, and the tubular member has a second end surface on one end part side and a first end surface on other end part side in an axial direction, and has a first opening connected to the hollow portion on at least the first end surface.

### CROSS REFERENCE TO RELATED APPLICATION

This is a continuation of International Application No. PCT/JP2024/019266 filed on May 24, 2024, and claims priority from Japanese Patent Application No. 2023-169863 filed on September 29, 2023, the entire disclosure of which is incorporated herein by reference.

### BACKGROUND OF THE INVENTION

### 1. Field of the Invention

The technology disclosed herein relates to a treatment system used with an endoscope.

### 2. Description of the Related Art

JP2022-548739A discloses a transseptal insertion device including: a sheath defining at least one hollow portion; one or more positioning balloons connected to a distal end of the sheath; and a puncturing section movably disposed within the hollow portion.

Japanese Patent No. 4653734 discloses a medical device for inducing controlled necrosis at a target region within a body, comprising: an elongated member having a proximal end part, a distal end part, and at least one hollow portion in fluid communication with a cooling fluid; a plurality of dissecting tips connected to a distal end of the elongated member and configured to contact and dissect tissue; and an ultrasound probe that controls a transmural depth of insertion of the plurality of dissecting tips into the tissue.

Japanese Patent No. 6674432 discloses a system including a probe for deploying at least one needle into tissue.

JP2021-525124A discloses an imaging component comprising: a shaft comprising a proximal end, a distal end, and a cavity; and an imaging transducer connected to the distal end of the shaft.

JP2020-518385A discloses a treatment probe comprising: a handle; a probe body; an imaging source connected to the probe body; and an ablation element connected to the probe body and configured to ablate a target tissue.

Japanese Patent No. 6243910 discloses a system including: an imaging component comprising an imaging handle section, an imaging shaft having a proximal end and a distal end, and an imaging transducer at the distal end of the imaging shaft; and a needle component comprising a needle handle section, a needle shaft having a distal end and a proximal end, and a needle structure reciprocably disposed on or within the needle shaft.

### SUMMARY OF THE INVENTION

The present disclosure provides the technology capable of improving the efficiency of an examination or a treatment using an endoscope.

An aspect of the technology disclosed herein relates to a treatment system comprising: a tubular member having a hollow portion therein and having, at one end part, a treatment part provided at an outer periphery of the hollow portion, in which the tubular member has a second end surface on one end part side and a first end surface on the other end part side in an axial direction, and has a first opening connected to the hollow portion on at least the first end surface.

Another aspect of the technology disclosed herein relates to a treatment method comprising: a first step of inserting an ultrasound probe into a hollow portion of a tubular member having the hollow portion therein and having, at one end part, a treatment part provided at an outer periphery of the hollow portion, in which an opening connected to the hollow portion is provided on an end surface on the other end part side in an axial direction; a second step of inserting the tubular member and the ultrasound probe into a subject in a state where an ultrasound transducer of the ultrasound probe is disposed at a position adjacent to the treatment part in the axial direction of the tubular member; a third step of aligning the ultrasound transducer with a target region of the subject based on an output of the ultrasound transducer obtained by moving the tubular member and the ultrasound probe within the subject; a fourth step of aligning the target region with the treatment part by moving the treatment part to the position of the ultrasound transducer in a state where the target region and ultrasound transducer are aligned; and a fifth step of treating the target region by driving the treatment part in a state where the target region and the treatment part are aligned.

According to the technology disclosed herein, the efficiency of the examination or the treatment using the endoscope can be improved.

### BRIEF DESCRIPTION OF THE DRAWINGS

FIG. 1 is a diagram illustrating a schematic configuration of an endoscope device 100 that is one aspect of the technology disclosed herein.

FIG. 2 is a schematic cross-sectional view illustrating a schematic configuration of a treatment system 20 that is used by being inserted from a treatment tool inlet port 112 of an endoscope 1 illustrated in FIG. 1 into a treatment tool channel of the endoscope 1.

FIG. 3 is an enlarged view of a range X illustrated in FIG. 2.

FIG. 4 is a cross-sectional view taken along line C-C illustrated in FIG. 3.

FIG. 5 is an enlarged view of a range Y illustrated in FIG. 2.

FIG. 6 is a diagram illustrating a state where a tubular member 31 is moved to a distal end side from a state illustrated in FIG. 3.

FIG. 7 is a diagram illustrating a state where an ultrasound probe 40 is moved to a proximal end side from the state illustrated in FIG. 6.

FIG. 8 is a schematic diagram illustrating a first modification example of the treatment system 20.

FIG. 9 is a schematic diagram illustrating a state where the ultrasound probe 40 and a treatment probe 30 are disposed at an endoscope distal end part 10C.

FIG. 10 is a schematic diagram illustrating a treatment method.

FIG. 11 is a schematic diagram illustrating the treatment method.

FIG. 12 is a schematic diagram illustrating the treatment method.

FIG. 13 is a schematic diagram illustrating a second modification example of the treatment system 20.

### DESCRIPTION OF THE PREFERRED EMBODIMENTS

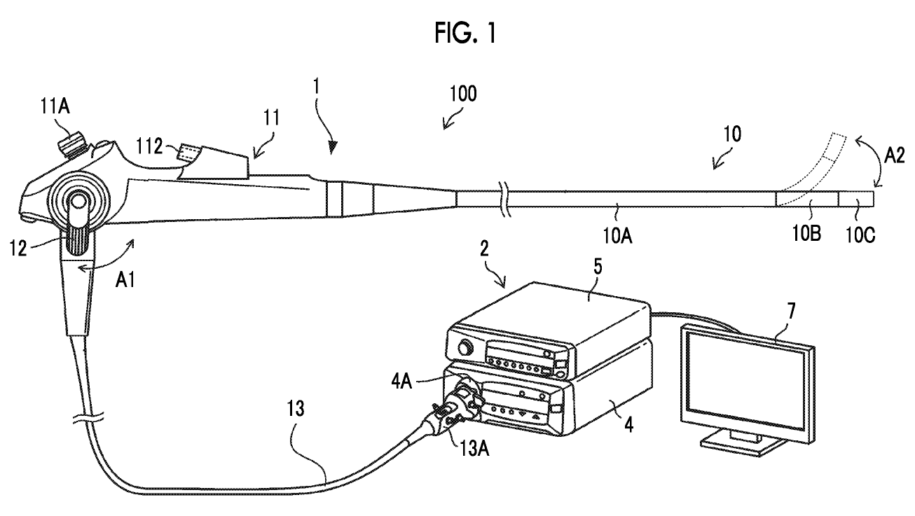

FIG. 1 is a diagram illustrating a schematic configuration of an endoscope device 100 that is one aspect of the technology disclosed herein. The endoscope device 100 comprises an endoscope 1, a body part 2 consisting of a light source device 4 and a processor device 5 to which the endoscope 1 is connected, and a display device 7 that displays a captured image captured by the endoscope 1, and the like. The endoscope 1 is a bronchoscope in the example of FIG. 1, but the present disclosure is not limited to this.

The endoscope 1 comprises an insertion part 10 that is an elongated tool extending in one direction and that is inserted into a subject, an operating part 11 that is provided at a proximal end part of the insertion part 10 and that is provided with an operation member for performing an observation mode switching operation, an imaging and storage operation, a forceps operation, a suction operation, and the like, an angle lever 12 that is provided adjacent to the operating part 11, and a universal cord 13 including a connector part 13A for attachably and detachably connecting the endoscope 1 to a connector connection part 4A of the light source device 4.

The operating part 11 is provided with a treatment tool inlet port 112 into which a treatment tool for collecting a biological tissue such as cells or polyps is introduced. Although not illustrated in FIG. 1, various channels such as a treatment tool channel and a suction channel into which the treatment tool introduced from the treatment tool inlet port 112 is inserted are provided inside the operating part 11 and the insertion part 10. The operating part 11 includes a suction button 11A and the like. A suction tube (not illustrated) is connected to the suction button 11A, and the suction tube is connected to a suction device. In a case where the suction button 11A is operated, a fluid or the like is suctioned from a distal end of the endoscope 1 to the suction tube and is then collected by the suction device.

The insertion part 10 includes a flexible part 10A having flexibility, a bendable part 10B provided at a distal end of the flexible part 10A, and an endoscope distal end part 10C that is harder than the flexible part 10A and that is provided at a distal end of the bendable part 10B. An imaging element and an imaging optical system are built in the endoscope distal end part 10C.

The bendable part 10B is configured to perform a bending operation in an up-down direction (A2 direction) by rotationally operating the angle lever 12 of the operating part 11 in an A1 direction, and the endoscope distal end part 10C can be directed in a desired direction by performing the bending operation of the bendable part 10B.

A light guide configured by bundling a plurality of optical fibers is provided inside the endoscope 1 from the endoscope distal end part 10C of the insertion part 10 to the connector part 13A. The light generated by the light source device 4 is introduced from the connector part 13A into the light guide and travels to the endoscope distal end part 10C, and is emitted to the subject from an illumination window provided in the endoscope distal end part 10C.

The configuration of the endoscope 1 is an example, and is not limited to the configuration illustrated in FIG. 1.

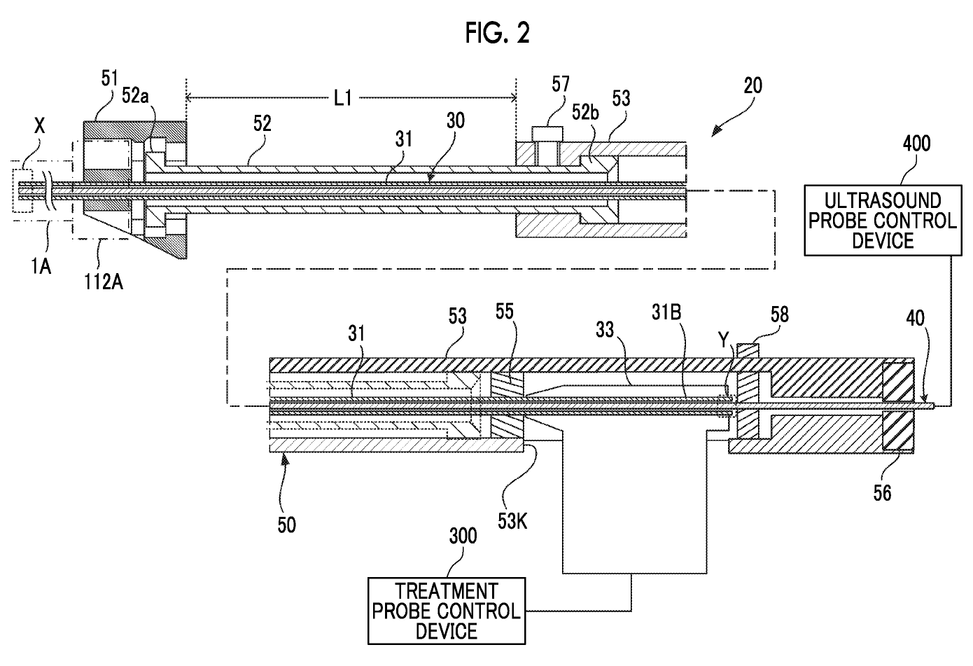

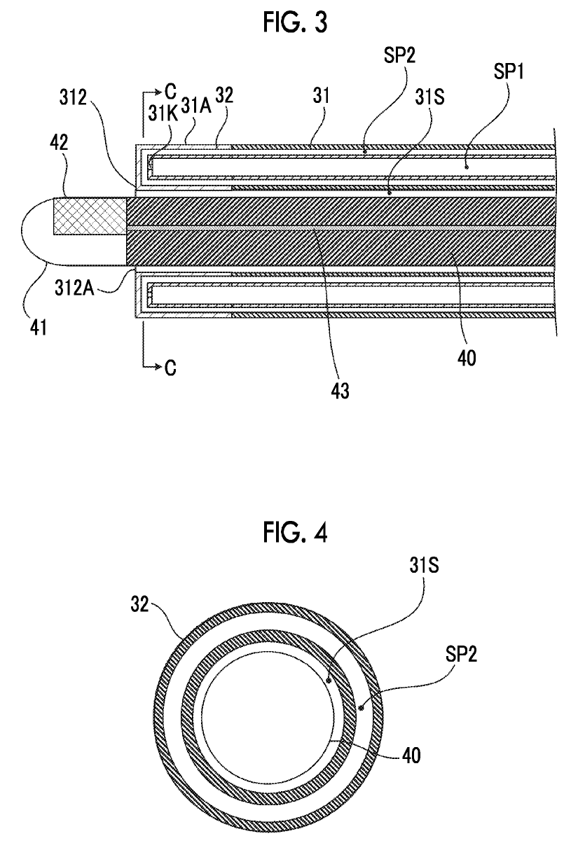

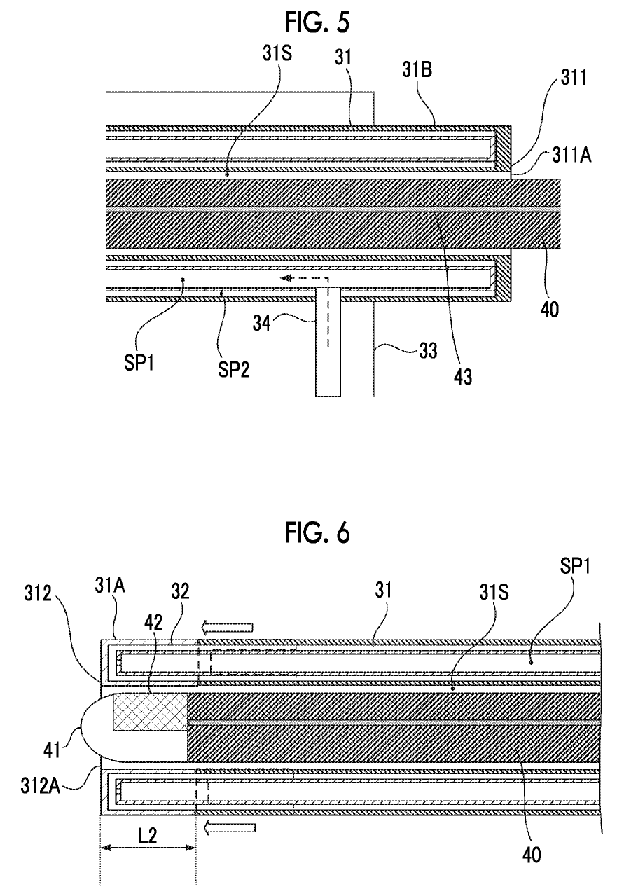

FIG. 2 is a schematic cross-sectional view illustrating a schematic configuration of a treatment system 20 that is used by being inserted from the treatment tool inlet port 112 of the endoscope 1 illustrated in FIG. 1 into the treatment tool channel of the endoscope 1. FIG. 3 is an enlarged view of a range X illustrated in FIG. 2. FIG. 4 is a cross-sectional view taken along line C-C illustrated in FIG. 3. FIG. 5 is an enlarged view of a range Y illustrated in FIG. 2.

The treatment system 20 comprises a treatment probe 30 that is an elongated treatment tool extending in one direction, an ultrasound probe 40 that is an elongated tool extending in one direction, and an operation handle 50 that is configured to support the treatment probe 30 and the ultrasound probe 40.

The treatment probe 30 includes a tubular member 31 having, for example, a cylindrical hollow portion 31S (see FIGS. 3 and 5) inside. The treatment probe 30 is a treatment tool that can freeze tissue of the subject by means of a treatment part 32 provided at a distal end part 31A (see FIG. 3) that is an end part of the tubular member 31 on one end side in an axial direction along a circumferential direction. As the treatment probe 30, a treatment probe in which the treatment part 32 is made of, for example, a metal and the treatment part 32 is cooled by using a Joule-Thomson effect is preferably used.

For example, a wall part of the tubular member 31 is provided with a first space SP1 having an annular cross section and a second space SP2 provided to surround the first space SP1. An opening 31K that connects the first space SP1 and the second space SP2 is provided in a portion of the treatment part 32 in the tubular member 31. The tubular member 31 is made of, for example, a resin or an elastomer in a portion other than the treatment part 32.

As illustrated in FIG. 5, the first space SP1 is connected to a supply pipe 34 for supplying a gas for cooling the treatment part 32. As illustrated in FIG. 2, a substantially T-shaped grip part 33 is fixed to a proximal end part 31B that is an end part of the tubular member 31 on the other end side in the axial direction, and the supply pipe 34 is provided inside the grip part 33. The supply pipe 34 is connected to a treatment probe control device 300. The treatment probe control device 300 performs various controls such as driving the treatment part 32 (injecting the gas for cooling).

The gas, which is supplied from the supply pipe 34, is supplied from the opening 31K to the second space SP2 through the first space SP1. During a process in which the gas flows from the first space SP1 to the second space SP2, the treatment part 32 is cooled by the Joule-Thomson effect. The gas that has flowed into the second space SP2 is discharged to an exhaust pipe (not illustrated). The treatment part 32 is provided in an annular shape around the hollow portion 31S, and thus the tissue in a range of 360 degrees around the distal end part 31A of the treatment probe 30 can be frozen by the treatment part 32.

As illustrated in FIG. 3, a distal end opening 312A connected to the hollow portion 31S is provided on a distal end surface 312 that is an end surface of the tubular member 31 on one end side in the axial direction. As illustrated in FIG. 5, a proximal end opening 311A connected to the hollow portion 31S is provided on a proximal end surface 311 that is an end surface of the tubular member 31 on the other end side in the axial direction.

The ultrasound probe 40 is used in a state of being inserted into the hollow portion 31S from the proximal end opening 311A of the treatment probe 30. It is preferable that an inner diameter of the tubular member 31 is configured to be greater than an outer diameter of the ultrasound probe 40.

As illustrated in FIG. 3, an ultrasound transmission/reception unit 42 is provided on a part of an outer peripheral surface of a substantially cylindrical distal end part 41 that is an end part of the ultrasound probe 40 on one end side. The ultrasound transmission/reception unit 42 is configured by arranging a plurality of ultrasound transducers, for example, and can transmit ultrasonic waves toward a radial direction in a specific range in a radial direction of the distal end part 41. The ultrasound transmission/reception unit 42 may be configured by a single ultrasound transducer.

A rotation shaft 43 having one end connected to the ultrasound transmission/reception unit 42 is provided inside the ultrasound probe 40. The rotation shaft 43 extends in a direction (axial direction of the ultrasound probe 40) intersecting (preferably orthogonal to) a transmission direction of the ultrasonic waves of the ultrasound transmission/reception unit 42, and the other end of the rotation shaft 43 is connected to a rotation member (not illustrated). In a case where the rotation member rotates, the rotation shaft 43 rotates, and the transmission range of the ultrasonic waves by the ultrasound transmission/reception unit 42 can be switched by rotating along the circumferential direction of the distal end part 41. As described above, since the ultrasound transmission/reception unit 42 is configured to rotate, an ultrasound image in a range of 360 degrees around the distal end part 41 in the subject can be obtained based on an echo received by the ultrasound transmission/reception unit 42.

The ultrasound transmission/reception unit 42 may be, for example, provided in an annular shape as viewed in the axial direction of the tubular member 31. Since the ultrasound transmission/reception unit 42 is provided in an annular shape, the ultrasound image in the range of 360 degrees around the distal end part 41 in the subject can be easily obtained based on the echo received by the ultrasound transmission/reception unit 42. In a case where the ultrasound transmission/reception unit 42 is provided in an annular shape, the rotation shaft 43 and the rotation member are not required.

A wiring group electrically connected to the ultrasound transmission/reception unit 42 is provided in the ultrasound probe 40. The wiring group is connected to an ultrasound probe control device 400 that performs control of the ultrasound transmission/reception unit 42 and generation and display of the ultrasound image.

The operation handle 50 has an elongated tubular shape in which a housing space in which a portion of the treatment probe 30 can be housed is provided. The operation handle 50 has a tubular connection part 51 that can be connected to an adapter 112A provided at the treatment tool inlet port 112 of the endoscope 1 by fitting or the like at a distal end thereof.

The operation handle 50 further includes a tubular shaft member 52 that is supported at one end of the connection part 51 and that extends to a side opposite to the connection part 51 side. In the shaft member 52, a flange part 52a is provided at an end part on the connection part 51 side, and a flange part 52b is provided at an end part on a side opposite to the connection part 51 side. The flange part 52a is inserted into the connection part 51, and is fixed inside the connection part 51. The tubular member 31 is inserted into the entire interior of the shaft member 52. The tubular member 31 on the distal end side of the flange part 52a of the shaft member 52 is inserted into the adapter 112A connected to the connection part 51 through the inside of the connection part 51.

In a case where the treatment system 20 is used, the treatment probe 30 led out to the outside of the connection part 51 is inserted into a treatment tool channel 1A of the endoscope 1 from the distal end part 31A through the adapter 112A, and the connection part 51 is connected to the adapter 112A in this state. The connection part 51 is configured to be connectable to the treatment tool inlet port 112 in a state where the tubular member 31 is inserted into the treatment tool inlet port 112 of the endoscope 1 from the distal end part 31A.

The operation handle 50 further comprises a tubular slider 53 that is slidably connected to an outer peripheral surface between the flange part 52a and the flange part 52b of the shaft member 52 in the axial direction of the shaft member 52. An opening is provided on an outer peripheral surface of the slider 53 at an end part on the connection part 51 side. A fixing member 57 such as a screw for fixing (locking) a relative position of the slider 53 to the shaft member 52 is inserted into the opening. In a state where the fixing member 57 is unlocked, the slider 53 is configured to move in a range of a distance L1 between a position illustrated in FIG. 2 at which an inner wall and the flange part 52b are in contact with each other and a position at which the end surface on the connection part 51 side is in contact with the connection part 51.

An opening 53K is provided on the outer peripheral surface of the slider 53 on a proximal end side of a position at which the flange part 52b is disposed in a state where the end surface on the connection part 51 side is in contact with the flange part 52a (a position of the shaft member 52 in this state is indicated by a broken line in the drawing). The grip part 33 is disposed at a position at which the opening 53K is provided. A portion of the grip part 33 protrudes to the outside from the opening 53K. The grip part 33 is fixed to the tubular member 31.

A support mechanism 55 that supports the proximal end part 31B of the tubular member 31 is provided inside the slider 53 adjacent to the distal end side (left side in FIG. 2) of the grip part 33. The support mechanism 55 includes a latch mechanism or the like, and supports the treatment probe 30 to be movable by a predetermined distance in the axial direction of the tubular member 31. For example, in a case where an operator grips the grip part 33 and moves the grip part 33 in the left direction in FIG. 3, the tubular member 31 (that is, the treatment probe 30) can be moved by a predetermined distance to the distal end side, and the state can be maintained.

A support member 56 that supports the ultrasound probe 40 is provided on the proximal end side of the grip part 33 in the slider 53. The support member 56 is made of an annular rubber or the like that fits into the opening provided on the proximal end surface of the slider 53, and the ultrasound probe 40 can be inserted. The support member 56 can fix and support the inserted ultrasound probe 40 by tightening a screw or the like (not illustrated) to narrow the hollow portion. The ultrasound probe 40 inserted into the support member 56 is inserted into the hollow portion 31S of the tubular member 31 supported by the support mechanism 55, and is inserted to the distal end of the tubular member 31.

A support mechanism 58 that supports the ultrasound probe 40 is provided between the grip part 33 and the support member 56 in the slider 53. The support mechanism 58 includes a latch mechanism or the like, and supports the ultrasound probe 40 to be movable by a predetermined distance in the axial direction. For example, in a case where the operator moves the support mechanism 58 in the right direction in FIG. 3, the ultrasound probe 40 can be moved by a predetermined distance to the proximal end side.

In the treatment system 20, it is preferable that the treatment probe 30 and the grip part 33 are attachably and detachably configured to the operation handle 50. In a case where the treatment system 20 is used, first, an assembly including the treatment probe 30 and the grip part 33 fixed to the treatment probe 30 is prepared, and the assembly is inserted into the slider 53 from the opening 53K. In this case, the treatment probe 30 is inserted from the distal end side into the slider 53, the shaft member 52, and the connection part 51 in this order, and most of the treatment probe 30 is exposed to the outside from the connection part 51. The treatment system 20 may have a configuration in which the operation handle 50, the treatment probe 30, and the grip part 33 are integrated and only the ultrasound probe 40 is attachable and detachable.

Then, the ultrasound probe 40 is inserted into the support member 56 from the distal end part 41 side, and the ultrasound probe 40 is inserted into the hollow portion 31S of the tubular member 31 supported by the support mechanism 55. Then, the distal end of the ultrasound probe 40 is inserted to the distal end of the tubular member 31 such that a positional relationship illustrated in FIG. 3 is obtained.

In the example of FIG. 3, the ultrasound transmission/reception unit 42 is located on a side opposite to the proximal end surface 311 side of the tubular member 31 in the axial direction with respect to the treatment part 32. As described above, the ultrasound probe 40 is supported by the support member 56 and the support mechanism 58 in a state where the ultrasound transmission/reception unit 42 can protrude from the distal end opening 312A to the outside of the hollow portion 31S. The treatment probe 30 can move in the axial direction in the slider 53 by means of the support mechanism 55. Therefore, in a case where the grip part 33 is operated, the treatment probe 30 can be moved by a predetermined distance to the distal end side without changing the position of the ultrasound probe 40.

FIG. 6 is a diagram illustrating a state where the grip part 33 is moved to the distal end side of the tubular member 31 from the state illustrated in FIG. 3. In a case where the grip part 33 is moved by a distance L2 to the distal end side from the position illustrated in FIG. 2, as illustrated in FIG. 6, a state where the ultrasound transmission/reception unit 42 retracts into the hollow portion 31S from the distal end opening 312A can be obtained. In addition, in the state illustrated in FIG. 6, in a state as viewed in the radial direction of the tubular member 31, the treatment part 32 and the ultrasound transmission/reception unit 42 overlap each other, and the positions of the treatment part 32 and the ultrasound transmission/reception unit 42 in the axial direction of the tubular member 31 substantially coincide with each other.

In the state illustrated in FIG. 6, a portion of the distal end part 41 protrudes from the distal end opening 312A, but the tubular member 31 may be movably supported by the support mechanism 55 such that the entire distal end part 41 retracts into the hollow portion 31S.

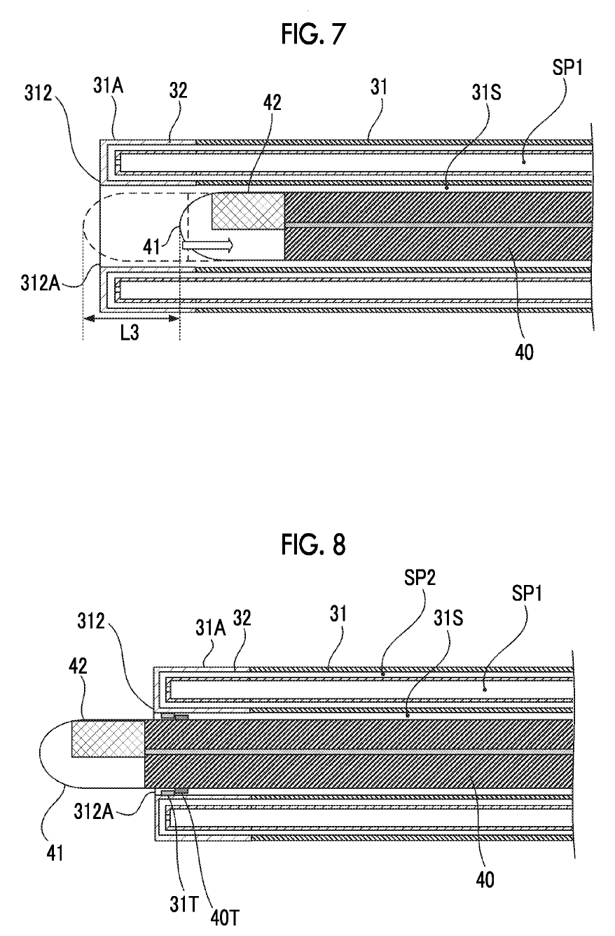

In addition, the ultrasound probe 40 can move in the axial direction in the slider 53 by means of the support mechanism 58. Therefore, in a case where the support mechanism 58 is operated, the ultrasound probe 40 can be moved by a distance L3 (see FIG. 7) to the proximal end side with respect to the treatment probe 30.

FIG. 7 is a diagram illustrating a state where the ultrasound probe 40 is moved to the proximal end side from the state illustrated in FIG. 6. In a case where the support mechanism 58 is operated and the ultrasound probe 40 is moved by the distance L3 to the proximal end side, as illustrated in FIG. 7, a state where the ultrasound transmission/reception unit 42 is located on the proximal end surface 311 side with respect to the treatment part 32 can be obtained. In the state illustrated in FIG. 7, it is preferable that the ultrasound probe 40 is movably supported by the support mechanism 58 such that the treatment part 32 and the ultrasound transmission/reception unit 42 do not overlap each other as viewed in the radial direction of the tubular member 31.

As described above, the support mechanism 55, the support member 56, and the support mechanism 58 support the tubular member 31 and the ultrasound probe 40 such that the relative position between the ultrasound transmission/reception unit 42 of the ultrasound probe 40 and the treatment part 32 of the treatment probe 30 can be changed.

In the initial state where the support mechanism 55 and the support mechanism 58 are not operated, the ultrasound transmission/reception unit 42 protrudes from the distal end opening 312A of the tubular member 31. A restriction part that restricts a protrusion amount of the ultrasound transmission/reception unit 42 from the distal end opening 312A in the initial state may be provided in the treatment system 20.

For example, as illustrated in FIG. 8, an annular protrusion part 31T is provided on an inner peripheral surface of the distal end part 31A of the tubular member 31, and a protrusion part 40T that can be brought into contact with the protrusion part 31T is provided on an outer peripheral surface of the distal end part 41 of the ultrasound probe 40 in the vicinity of the distal end part 41. In a case where the ultrasound probe 40 is inserted into the hollow portion 31S of the tubular member 31, the protrusion amount of the ultrasound transmission/reception unit 42 from the hollow portion 31S is maximized in a state where the protrusion part 31T and the protrusion part 40T are in contact with each other, so that the protrusion amount can be restricted. In the state illustrated in FIG. 8, the ultrasound probe 40 is fixed by the support member 56, so that the positional relationship between the treatment probe 30 and the ultrasound probe 40 can be controlled to a desired position. In the configuration illustrated in FIG. 8, the protrusion part 31T and the protrusion part 40T constitute a first restriction part.

In addition, in the operation handle 50, a support position of the tubular member 31 may be fixed (the support mechanism 55 supports the tubular member 31 to be immovable in the axial direction), and only the ultrasound probe 40 may be supported to be movable in the axial direction of the tubular member 31. Even with such a configuration, the transition from the state illustrated in FIG. 3 to the state illustrated in FIG. 6 and the transition from the state illustrated in FIG. 6 to the state illustrated in FIG. 7 can be performed by moving the ultrasound probe 40 with respect to the treatment probe 30.

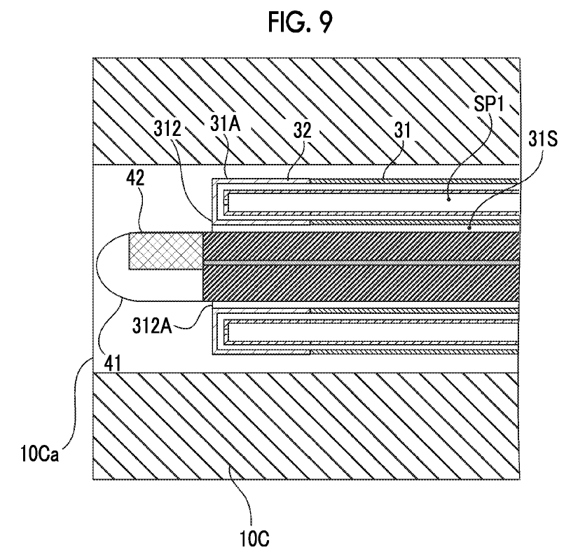

After the ultrasound probe 40 is inserted into the treatment probe 30 to obtain the state illustrated in FIG. 3 (state where the ultrasound transmission/reception unit 42 protrudes from the distal end opening 312A), the treatment probe 30 in a state where the ultrasound probe 40 is inserted is inserted into the treatment tool inlet port 112 from the distal end side, and then the connection part 51 is connected to the treatment tool inlet port 112. In this state, as illustrated in FIG. 9, the treatment probe 30 and the ultrasound probe 40 retract into the inside of the endoscope distal end part 10C with respect to a treatment tool outlet port 10Ca formed on the end surface of the endoscope distal end part 10C. It is also possible to change the structure of the operation handle 50 such that the treatment probe 30 is inserted into the treatment tool inlet port 112 from the distal end side in a state where the distal end of the ultrasound probe 40 is inserted to the distal end of the tubular member 31 so that the positional relationship illustrated in FIG. 6 is obtained.

The slider 53 supports the support mechanism 55 and the support mechanism 58. Therefore, in a case where the slider 53 is moved along the shaft member 52 in a state where the support mechanism 55 and the support mechanism 58 are not operated, the treatment probe 30 and the ultrasound probe 40 can be moved at the same time from the state illustrated in FIG. 9. As a result, the treatment probe 30 and the ultrasound probe 40 can protrude from the treatment tool outlet port 10Ca or can retract into the treatment tool outlet port 10Ca.

As described above, a movement mechanism that moves the tubular member 31 and the ultrasound probe 40 in the axial direction of the tubular member 31 in a state where the relative position between the ultrasound transmission/reception unit 42 and the treatment part 32 is maintained is configured by the shaft member 52, the slider 53, the support mechanism 55, the support member 56, and the support mechanism 58.

The slider 53 can be moved only between a position in contact with the connection part 51 and a position in contact with the flange part 52b. Therefore, the protrusion amounts of the tubular member 31 and the ultrasound probe 40 from the treatment tool outlet port 10Ca are restricted by the shaft member 52, the slider 53, and the connection part 51. The shaft member 52, the slider 53, and the connection part 51 constitute a second restriction part.

An example of a treatment method using the endoscope 1 and the treatment system 20 configured as above will be described. First, the operator inserts the insertion part 10 of the endoscope 1 into a bronchus of the subject, and moves the endoscope distal end part 10C to a desired region. Then, the treatment probe 30 and the ultrasound probe 40 of the treatment system 20 prepared as described above are inserted into the treatment tool inlet port 112, and the connection part 51 is connected to the treatment tool inlet port 112. In addition, the treatment system 20 may be mounted on the endoscope 1 before the endoscope 1 is inserted into the subject.

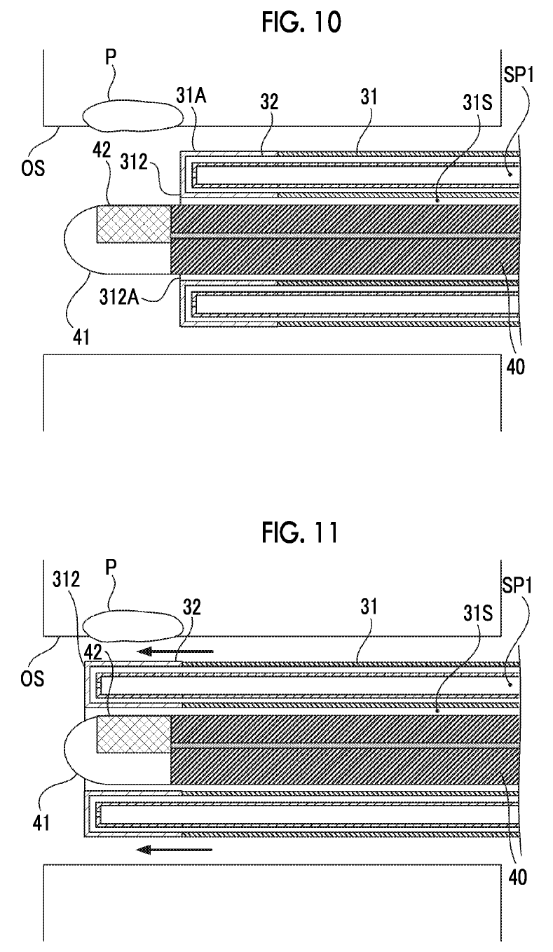

Next, the operator slides the slider 53 to protrude the treatment probe 30 and the ultrasound probe 40 from the treatment tool outlet port 10Ca and to advance the treatment probe 30 and the ultrasound probe 40 in the bronchus, and aligns the ultrasound transmission/reception unit 42 with a target region P of a bronchus OS while checking the ultrasound image generated based on the output of each ultrasound transducer of the ultrasound transmission/reception unit 42 (see FIG. 10). In the state illustrated in FIG. 10, the target region P and the ultrasound transducer are aligned with each other. The treatment probe 30 and the ultrasound probe 40 in the state illustrated in FIG. 6 may protrude from the treatment tool outlet port 10Ca and may be advanced in the bronchus, and may be changed to the state illustrated in FIG. 3 after reaching a desired position to align the ultrasound transmission/reception unit 42 with the target region P of the bronchus OS.

Next, in the state illustrated in FIG. 10, the operator slides the grip part 33 to the distal end side to move the tubular member 31 to the distal end part 31A side while maintaining the position of the ultrasound transmission/reception unit 42, as illustrated in FIG. 11, to align the ultrasound transmission/reception unit 42 with the treatment part 32.

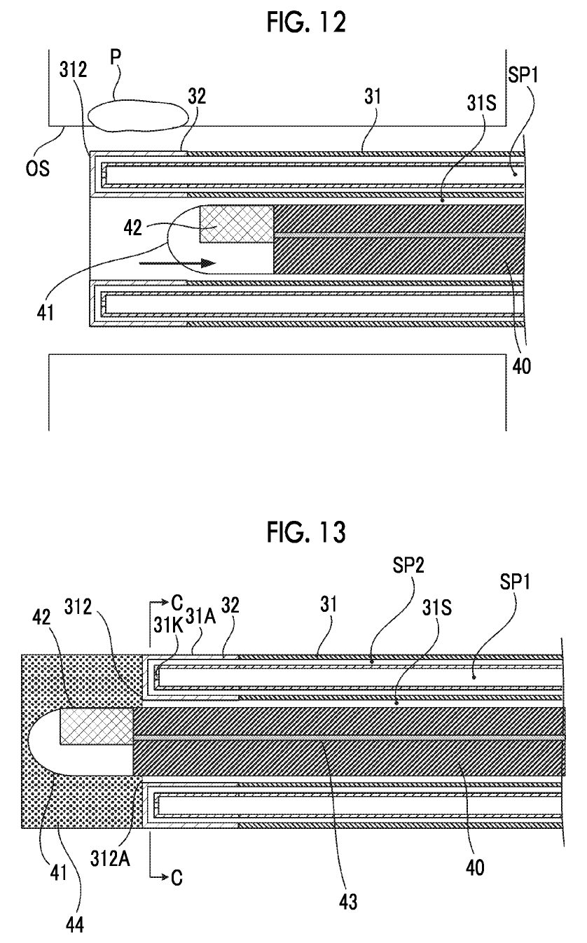

Next, the operator operates the support mechanism 58 to move the ultrasound probe 40 to the proximal end side, as illustrated in FIG. 12.

Next, the operator drives the treatment part 32 to treat the target region P. That is, the treatment part 32 is cooled to freeze the target region P. Thereafter, the operator removes the connection part 51 from the treatment tool inlet port 112 while maintaining the positional relationship between the treatment probe 30 and the ultrasound probe 40 illustrated in FIG. 12, and pulls out the treatment probe 30 and the ultrasound probe 40 from the treatment tool inlet port 112 to collect the frozen tissue. After the target region P is frozen, the insertion part 10 of the endoscope 1 may be pulled out from the subject, and the insertion part 10, the treatment probe 30, and the ultrasound probe 40 may be pulled out at the same time.

As described above, with the treatment system 20, even in a narrow region such as the bronchus that cannot be observed by the endoscope 1, the target region P can be searched for by the ultrasound probe 40, and the target region P can be treated (tissue can be collected) by the treatment part 32 without removing the ultrasound probe 40 from the endoscope 1.

For example, it is not necessary to perform work (work of replacing the ultrasound probe and the treatment probe) of searching for the target region P by inserting the ultrasound probe into the treatment tool channel of the endoscope 1, pulling out the ultrasound probe from the treatment tool channel, inserting the treatment probe into the treatment tool channel to advance the treatment part to the position of the target region P, and performing the treatment in this state. Therefore, the treatment can be simply and efficiently carried out.

In addition, in the state illustrated in FIG. 11 in which the tubular member 31 is slid by the support mechanism 55, the ultrasound transmission/reception unit 42 and the treatment part 32 are disposed at substantially the same position. Therefore, the treatment part 32 can be accurately aligned with the target region P, and the treatment can be accurately performed on the target region P confirmed in the ultrasound image. In addition, compared to a method of searching for the target region using radiation, the cost of the treatment can be reduced while eliminating the effect on the subject because the ultrasound is used.

In addition, with the treatment system 20, as illustrated in FIG. 12, the treatment part 32 can be driven in a state where the ultrasound transmission/reception unit 42 is not disposed inside the treatment part 32. Therefore, heat generated in association with cooling of the treatment part 32 can be prevented from affecting the ultrasound transmission/reception unit 42, and the durability of the ultrasound probe 40 can be improved.

With the treatment system 20, since the ultrasound probe 40 can be inserted and pulled out with respect to the treatment probe 30, a commercially available ultrasound probe 40 can be used as the ultrasound probe 40, and the implementation cost of the system can be reduced. In the treatment system 20, the operation handle 50 and the ultrasound probe 40 may be washed and reused, and the treatment probe 30 may be a single-use disposable device. Alternatively, the ultrasound probe 40 may be washed, disinfected, and reused, and the operation handle 50 and the treatment probe 30 may be a single-use disposable device.

In the treatment system 20, it is not essential that the relative position between the treatment probe 30 and the ultrasound probe 40 is changeable. The treatment probe 30 and the ultrasound probe 40 may be fixed in a state of having the positional relationship as illustrated in FIG. 3. With this configuration, after the ultrasound transmission/reception unit 42 and the target region P are aligned with each other, the slider 53 is slid to move the treatment probe 30 and the ultrasound probe 40 to the left in FIG. 3, the treatment part 32 is moved to the position at which the ultrasound transmission/reception unit 42 is present, and the treatment part 32 is driven in this state to freeze the target region P. In this configuration, by providing a mechanism (for example, adding gradations or the like to the slider 53) that can move the slider 53 to the distal end side by the same distance as the distance between the treatment part 32 and the ultrasound transmission/reception unit 42, more accurate treatment can be performed on the target region P. In addition, with this configuration, for example, as illustrated in FIG. 13, a distal end member 44 made of a material capable of propagating the ultrasound may be used to close the distal end opening 312A. The distal end part 41 illustrated in FIG. 13 is inserted into the hollow portion of the distal end member 44 and is fitted or fixed to the distal end member 44 by adhesion or the like.

In the configuration example illustrated in FIG. 13, it is assumed that the treatment system 20 is sold in a state where the treatment probe 30, the operation handle 50, and the ultrasound probe 40 are assembled. However, the distal end member 44 may be configured as an independent part, and the distal end part 41 may be fitted with this part after the ultrasound probe 40 is inserted into the treatment probe 30 as illustrated in FIG. 3, so that the configuration illustrated in FIG. 13 may be obtained.

The treatment probe 30 described above is not limited to the treatment probe 30 that can freeze and collect the tissue, and one that collects the tissue by another method can also be used. For example, the treatment part 32 can be changed to a brush-shaped treatment part. In this case, for example, a configuration need only be adopted in which the brush is folded in a normal state and the brush is opened to a position at which the brush can contact the target region P in a treatment state.

Further, the treatment part 32 can be forceps. In this case, for example, a configuration need only be adopted in which the forceps are folded in a normal state and the forceps are opened to a position at which the forceps can contact the target region P in a treatment state.

In the above description, the ultrasound transmission/reception unit 42 is configured to rotate inside the ultrasound probe 40, but the present disclosure is not limited to this. For example, the entire ultrasound probe 40 may be configured to rotate about the axis.

As described above, this specification sets forth at least the following matters. Hereinafter, components corresponding to the above embodiments are indicated in parentheses; however, the invention is not limited to this.

(1)

A treatment system (treatment system 20) comprising: a tubular member (tubular member 31) having a hollow portion (hollow portion 31S) therein and having, at one end part (distal end part 31A), a treatment part (treatment part 32) provided at an outer periphery of the hollow portion, in which the tubular member has a second end surface (distal end surface 312) on one end part side and a first end surface (proximal end surface 311) on the other end part (proximal end part 31B) side in an axial direction, and has a first opening (proximal end opening 311A) connected to the hollow portion on at least the first end surface.

(2)

The treatment system according to (1), in which a second opening (distal end opening 312A) connected to the hollow portion is provided on the second end surface of the tubular member.

(3)

The treatment system according to (1) or (2), further comprising: a device (operation handle 50) including a first support part (support mechanism 55) that supports the other end part of the tubular member, and a connection part (connection part 51) that is connectable to a treatment tool inlet port (treatment tool inlet port 112) of an endoscope in a state where the tubular member is inserted into the treatment tool inlet port from the one end part side, in which the device includes a second support part (support member 56, support mechanism 58) that supports an ultrasound probe (ultrasound probe 40) inserted into the hollow portion.

(4)

The treatment system according to (3), in which the first support part and the second support part support the tubular member and the ultrasound probe such that a relative position between an ultrasound transducer (ultrasound transmission/reception unit 42), that is provided at a distal end part (distal end part 41) of the ultrasound probe on a side of the second end surface, and the treatment part is changeable.

(5)

The treatment system according to (4), in which the relative position includes a first state (state illustrated in FIG. 3) where the ultrasound transducer is located, with respect to the treatment part, on a side opposite to a side of the first end surface.

(6)

The treatment system according to (5), in which a second opening (distal end opening 312A) connected to the hollow portion is provided on the second end surface of the tubular member, and the first state is a state where the ultrasound transducer protrudes from the second opening.

(7)

The treatment system according to (6), further comprising: a first restriction part that restricts a protrusion amount of the ultrasound transducer from the second opening.

(8)

The treatment system according to (6), in which the relative position includes a second state (state illustrated in FIG. 6 or state illustrated in FIG. 7) where the ultrasound transducer is located on the side of the first end surface with respect to the second opening.

(9)

The treatment system according to (8), in which the second state is a state (state illustrated in FIG. 7) where the ultrasound transducer is located, with respect to the treatment part, on the side of the first end surface.

(10)

The treatment system according to any one of (3) to (9), in which the device includes a movement mechanism that moves the tubular member and the ultrasound probe in the axial direction in a state where the device is connected to the treatment tool inlet port and in a state (state illustrated in FIG. 3) where a relative position between an ultrasound transducer (ultrasound transmission/reception unit 42), that is provided at a distal end part (distal end part 41) of the ultrasound probe on a side of the second end surface, and the treatment part is maintained.

(11)

The treatment system according to (10), in which the movement mechanism includes a second restriction part that restricts protrusion amounts of the tubular member and the ultrasound probe from a treatment tool outlet port (treatment tool outlet port 10Ca) of a distal end surface of the endoscope.

(12)

The treatment system according to any one of (1) to (11), further comprising: an ultrasound probe (ultrasound probe 40) that is inserted into the hollow portion in a state where a distal end part (distal end part 41) provided with an ultrasound transducer faces a side of the second end surface.

(13)

The treatment system according to (12), in which the ultrasound transducer (ultrasound transmission/reception unit 42) of the ultrasound probe is configured to rotate about a rotation shaft (rotation shaft 43) extending in a direction intersecting a transmission direction of ultrasonic waves of the ultrasound transducer.

(14)

The treatment system according to (13), in which a second opening (distal end opening 312A) connected to the hollow portion is provided on the second end surface of the tubular member, and the ultrasound probe is configured to switch between a state (state illustrated in FIG. 3) where the ultrasound transducer protrudes from the second opening and a state (states illustrated in FIGS. 6 and 7) where the ultrasound transducer retracts into the hollow portion from the second opening.

(15)

A treatment method comprising: a first step of inserting an ultrasound probe (ultrasound probe 40) into a hollow portion (hollow portion 31S) of a tubular member (tubular member 31) having the hollow portion therein and having, at one end part (distal end part 31A), a treatment part (treatment part 32) provided at an outer periphery of the hollow portion, in which an opening (proximal end opening 311A) connected to the hollow portion is provided on an end surface (proximal end surface 311) on the other end part (proximal end part 31B) side in an axial direction; a second step of inserting the tubular member and the ultrasound probe into a subject in a state where an ultrasound transducer (ultrasound transmission/reception unit 42) of the ultrasound probe is disposed at a position adjacent to the treatment part in the axial direction of the tubular member; a third step of aligning the ultrasound transducer with a target region (target region P) of the subject based on an output of the ultrasound transducer obtained by moving the tubular member and the ultrasound probe within the subject; a fourth step of aligning the target region with the treatment part by moving the treatment part to the position of the ultrasound transducer in a state where the target region and ultrasound transducer are aligned; and a fifth step of treating the target region by driving the treatment part in a state where the target region and the treatment part are aligned.

(16)

The treatment method according to (15), in which the fifth step includes a step of moving the ultrasound transducer toward the other end part side of the treatment part in a state where the target region and the treatment part are aligned, and the treatment part is driven after the step.

(17)

The treatment method according to (15), in which the position adjacent to the treatment part in the second step is a position adjacent to a side opposite to the other end part side.

(18)

The treatment method according to any one of (15) to (17), in which the treatment part is capable of freezing tissue, and the treatment method further comprises a sixth step of moving the ultrasound probe and the tubular member to collect the frozen tissue after the fifth step.

While various embodiments have been described above, it goes without saying that the present invention is not limited to such examples. It will be apparent to those skilled in the art that various modifications and alterations can be conceived within the scope set forth in the claims, and such modifications are understood to fall within the technical scope of the invention. Further, the respective components of the embodiments described above may be combined arbitrarily, provided such combinations do not depart from the gist of the invention.

This application is based on Japanese Patent Application No. 2023-169863, filed on September 29, 2023, the contents of which are incorporated herein by reference.

### EXPLANATION OF REFERENCES

1: endoscope

SP1: first space

SP2: second space

1A: treatment tool channel

2: body part

4: light source device

4A: connector connection part

5: processor device

7: display device

10: insertion part

10A: flexible part

10B: bendable part

10C: endoscope distal end part

10Ca: treatment tool outlet port

11: operating part

11A: suction button

12: angle lever

13: universal cord

13A: connector part

20: treatment system

30: treatment probe

31: tubular member

31A: distal end part

31B: proximal end part

31K, 53K: opening

31S: hollow portion

31T, 40T: protrusion

32: treatment part

33: grip part

34: supply pipe

40: ultrasound probe

41: distal end part

42: ultrasound transmission/reception unit

43: rotation shaft

44: distal end member

50: operation handle

51: connection part

52: shaft member

52a, 52b: flange part

53: slider

55, 58: support mechanism

56: support member

57: fixing member

100: endoscope device

112: treatment tool inlet port

112A: adapter

300: treatment probe control device

311: proximal end surface

311A: proximal end opening

312: distal end surface

312A: distal end opening

400: ultrasound probe control device

What is claimed is:

1. A treatment system comprising:
a tubular member having a hollow portion therein and having, at one end part, a treatment part provided at an outer periphery of the hollow portion,
wherein the tubular member has a second end surface on one end part side and a first end surface on other end part side in an axial direction, and has a first opening connected to the hollow portion on at least the first end surface.

2. The treatment system according to claim 1,
wherein a second opening connected to the hollow portion is provided on the second end surface of the tubular member.

3. The treatment system according to claim 1, further comprising:
a device including a first support part that supports the other end part of the tubular member, and a connection part that is connectable to a treatment tool inlet port of an endoscope in a state where the tubular member is inserted into the treatment tool inlet port from the one end part side,
wherein the device includes a second support part that supports an ultrasound probe inserted into the hollow portion.

4. The treatment system according to claim 3,
wherein the first support part and the second support part support the tubular member and the ultrasound probe such that a relative position between an ultrasound transducer, that is provided at a distal end part of the ultrasound probe on a side of the second end surface, and the treatment part is changeable.

5. The treatment system according to claim 4,
wherein the relative position includes a first state where the ultrasound transducer is located, with respect to the treatment part, on a side opposite to a side of the first end surface.

6. The treatment system according to claim 5,
wherein a second opening connected to the hollow portion is provided on the second end surface of the tubular member, and
the first state is a state where the ultrasound transducer protrudes from the second opening.

7. The treatment system according to claim 6, further comprising:
a first restriction part that restricts a protrusion amount of the ultrasound transducer from the second opening.

8. The treatment system according to claim 6,
wherein the relative position includes a second state where the ultrasound transducer is located on the side of the first end surface with respect to the second opening.

9. The treatment system according to claim 8,
wherein the second state is a state where the ultrasound transducer is located, with respect to the treatment part, on the side of the first end surface.

10. The treatment system according to claim 3,
wherein the device includes a movement mechanism that moves the tubular member and the ultrasound probe in the axial direction in a state where the device is connected to the treatment tool inlet port and in a state where a relative position between an ultrasound transducer, that is provided at a distal end part of the ultrasound probe on a side of the second end surface, and the treatment part is maintained.

11. The treatment system according to claim 10,
wherein the movement mechanism includes a second restriction part that restricts protrusion amounts of the tubular member and the ultrasound probe from a treatment tool outlet port of a distal end surface of the endoscope.

12. The treatment system according to claim 1, further comprising:
an ultrasound probe that is inserted into the hollow portion in a state where a distal end part provided with an ultrasound transducer faces a side of the second end surface.

13. The treatment system according to claim 12,
wherein the ultrasound transducer of the ultrasound probe is configured to rotate about a rotation shaft extending in a direction intersecting a transmission direction of ultrasonic waves of the ultrasound transducer.

14. The treatment system according to claim 13,
wherein a second opening connected to the hollow portion is provided on the second end surface of the tubular member, and
the ultrasound probe is configured to switch between a state where the ultrasound transducer protrudes from the second opening and a state where the ultrasound transducer retracts into the hollow portion from the second opening.

15. The treatment system according to claim 2, further comprising:
a device including a first support part that supports the other end part of the tubular member, and a connection part that is connectable to a treatment tool inlet port of an endoscope in a state where the tubular member is inserted into the treatment tool inlet port from the one end part side,
wherein the device includes a second support part that supports an ultrasound probe inserted into the hollow portion.

16. The treatment system according to claim 2, further comprising:
an ultrasound probe that is inserted into the hollow portion in a state where a distal end part provided with an ultrasound transducer faces a side of the second end surface.
